(19) United States
(12) Patent Application Publication
(10) Pub. No.: US 2025/0087334 A1
(43) Pub. Date: Mar. 13, 2025

(54) MOOD-TIMING-DEPENDENT THERAPEUTIC PATH ROUTING

(71) Applicant: WOEBOT LABS, INC., San Francisco, CA (US)

(72) Inventors: Alison DARCY, San Francisco, CA (US); Jade 2. DANIELS, San Francisco, CA (US)

(21) Appl. No.: 18/799,350

(22) Filed: Aug. 9, 2024

Related U.S. Application Data
(60) Provisional application No. 63/537,161, filed on Sep. 7, 2023.
(60) Provisional application No. 63/537,164, filed on Sep. 7, 2023.

(51) Int. Cl. G16H 20/70; A61B 5/16; H04L 51/02

(52) U.S. Cl. CPC G16H 20/70; A61B 5/165; H04L 51/02

(57) ABSTRACT

Systems and methods are disclosed for managing therapeutic path routing based on mood evaluation data. In a therapy-providing system, therapy can be provided to a user according to preset therapeutic paths, each of which can lead the user through different therapeutic exercises and experiences. Mood evaluation data can be collected periodically or in response to certain therapy milestone events. Depending on the mood evaluation data, optionally including the time elapsed since the mood evaluation data was collected, a particular therapeutic path can be selected out of a set of possible therapeutic paths.

### CROSS-REFERENCE TO RELATED APPLICATIONS

The present application claims the benefit of U.S. Provisional Patent Application No. 63/537,161 filed Sep. 7, 2023 and entitled “MOOD-TIMING-DEPENDENT CONVERSATION PATH ROUTING” and claims the benefit of U.S. Provisional Patent Application No. 63/537,164 filed Sep. 7, 2023 and entitled “MOOD-TIMING-DEPENDENT THERAPEUTIC PATH ROUTING,” the disclosures of which are hereby incorporated by reference in their entirety.

### TECHNICAL FIELD

The present disclosure relates to mood feedback systems generally and more specifically to automated therapeutic and conversational systems based on mood determination.

### BACKGROUND

Mental health problems are widespread, persistent, and stigmatized, with depression now the leading cause of disability worldwide. There are not enough trained professionals to deliver appropriate screening, diagnosis, and treatment. It can be difficult for individuals suffering from mental health problems to obtain treatment for a number of reasons, including difficulty in finding a provider, difficulty in paying for treatment, difficulty in self-identifying a need for treatment, and other reasons. Even if an individual finds a suitable provider and makes an effort to obtain treatment, that individual may need to wait long periods of time before an initial session due to the provider's schedule. Additionally, an individual's success in certain types of therapy, such as Cognitive-behavioral Therapy (CBT), depends strongly on the individual's ability to continue practicing certain exercises and data collection between sessions.

Self-directed treatment methods generally involve following exercises provided by a provider or presented in a book. Certain chatbots exist to mimic human-to-human therapy or assist a user in following self-directed treatment, but such chatbots follow fixed scripts, are unable to adapt to a user's individual needs, and are unable to establish a strong therapeutic bond like those established between patient and human provider.

In the field of human psychology, empathy is a powerful and important tool to engage a patient and provide effective therapy. In human-human interactions, empathy is a core technique for establishing and maintaining a therapeutic alliance between the therapist and client, which can be important to gain the client's trust and participation in the therapy. However, existing chatbots are unable to believably mimic empathetic communications, such as by engaging the user in insensitive ways and/or at insensitive times, which can lead to a breakdown of trust and an overall failure of the therapeutic alliance and human-computer relationship, which can be detrimental to the efficacy of the therapy.

There is a need for a tool to provide automated, personalized therapy to individuals. There is a need for a tool that can facilitate self-directed treatment in a fashion that is personalized to the individual. There is a need for a tool that can provide personalized therapy to individuals that dynamically changes according to the needs of the individual. There is a need for a tool that can engage users in an empathetic fashion.

### SUMMARY

The term embodiment and like terms are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, supplemented by this summary. This summary is a high-level overview of various aspects of the disclosure and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings and each claim.

Embodiments of the present disclosure include a method comprising initiating a mood evaluation request. The method further includes receiving user input indicative of current mood evaluation data in response to the mood evaluation request. The method further includes selecting a therapeutic path from a set of possible therapeutic paths based at least in part on the current mood evaluation data. At least a first therapeutic path of the set of possible therapeutic paths is selected when the current mood evaluation data is indicative of a first mood and a second therapeutic path of the set of possible therapeutic paths is selected when the current mood evaluation data is indicative of a second mood. The method further comprises generating a communication based at least in part on the selected therapeutic path. The method further comprises transmitting the communication, wherein the communication, when received, is presented via an output device.

In some cases, initiating the mood evaluation request occurs in response to completing a therapy milestone. In some cases, initiating the mood evaluation request automatically occurs after a period of time following a previous mood evaluation request. In some cases, selecting a therapeutic path from the set of possible therapeutic paths based on the current mood evaluation data further includes generating a current mood score based at least in part on the current mood evaluation data; and selecting the selected therapeutic path based at least in part on the current mood score.

In some cases, the method further comprises identifying a subsequent therapy milestone after transmitting the communication and determining, based at least in part on the current mood evaluation data and in response to identifying the subsequent therapy milestone, whether to initiate a subsequent mood evaluation request. In some cases, identifying the subsequent therapy milestone includes identifying ceasing of a therapeutic exercise. In some cases, transmitting the communication includes initiating the therapeutic exercise or suggesting initiation of the therapeutic exercise.

In some cases, determining whether to initiate the subsequent mood evaluation request includes deciding to initiate the subsequent mood evaluation request, the method further comprising: initiating the subsequent mood evaluation request; receiving subsequent user input indicative of subsequent mood evaluation data in response to the subsequent mood evaluation request; determining mood change information using the current mood evaluation data and the subsequent mood evaluation data; storing the mood change information in association with the therapeutic exercise; selecting a subsequent therapeutic path from a set of possible subsequent therapeutic paths based at least in part on the mood change information; generating a subsequent communication based at least in part on the selected subsequent therapeutic path; and transmitting the subsequent communication, wherein the subsequent communication, when received, is presented via the output device.

In some cases, the method further comprises determining an elapsed time since receiving the user input indicative of the current mood evaluation data, wherein determining whether to initiate the subsequent mood evaluation request is further based at least in part on the elapsed time. In some cases, determining whether to initiate the subsequent mood evaluation request includes determining to initiate the subsequent mood evaluation request when the current mood evaluation data is indicative of a negative mood and wherein the elapsed time is lower than a threshold elapsed time.

In some cases, receiving the user input indicative of current mood evaluation data includes storing the current mood evaluation data along with a timestamp of when the user input was received. In some cases, the method further comprises determining an elapsed time since receiving the user input indicative of the current mood evaluation data, wherein selecting the therapeutic path is further based at least in part on the elapsed time. In some cases, the method further comprises identifying a therapy milestone; receiving therapeutic data associated with the therapy milestone; and determining an elapsed time between identifying the therapy milestone and receiving the user input, wherein selecting the therapeutic path is further based at least in part on the therapeutic data and the elapsed time. In some cases, the therapy milestone is completion of a therapeutic exercise and wherein the therapeutic data is based at least in part on a past user input received during the therapeutic exercise. In some cases, the method further comprises initiating a chatbot session, wherein receiving the user input and transmitting the communication occur via the chatbot session. In some cases, the current mood evaluation data is indicative of i) a positive mood, ii) a negative mood, or iii) a neutral mood.

Embodiments of the present disclosure include a system comprising one or more data processors; and a non-transitory computer-readable storage medium containing instructions which, when executed on the one or more data processors, cause the one or more data processors to perform the above method(s).

Embodiments of the present disclosure include a computer-program product tangibly embodied in a non-transitory machine-readable storage medium, including instructions configured to cause a data processing apparatus to perform the above method(s).

### BRIEF DESCRIPTION OF THE DRAWINGS

The specification makes reference to the following appended figures, in which use of like reference numerals in different figures is intended to illustrate like or analogous components.

FIG. 1 is a schematic diagram depicting a computing environment, according to certain aspects of the present disclosure.

FIG. 2 is a flowchart depicting a process for selecting a therapeutic path, according to certain aspects of the present disclosure.

FIG. 3 is a set of tables depicting timestamped mood evaluation information and timestamped therapy milestones, according to certain aspects of the present disclosure.

FIG. 4 is a schematic diagram depicting an example conversation path on a graphical user interface for a positive mood evaluation response, according to certain aspects of the present disclosure.

FIG. 5 is a schematic diagram depicting an example conversation path on a graphical user interface for a negative mood evaluation response, according to certain aspects of the present disclosure.

FIG. 6 is a flowchart depicting a process for selecting a conversation path based on mood evaluation information, according to certain aspects of the present disclosure.

FIG. 7 is a block diagram depicting an example system architecture for implementing certain features and processes of the present disclosure.

### DETAILED DESCRIPTION

Certain aspects and features of the present disclosure relate to systems and methods for managing conversation path routing based on mood evaluation data. Mood evaluation data can be collected periodically or in response to certain events. Depending on the mood evaluation data, automated conversation paths may be followed to engage a user more efficiently on the user's current mood. One example of such a conversation path is therapeutic path routing based on mood evaluation data. In a therapy-providing system, therapy can be provided to a user according to preset therapeutic paths, each of which can lead the user through different therapeutic exercises and experiences. Mood evaluation data can be collected periodically or in response to certain therapy milestone events. Depending on the mood evaluation data, in some cases including the time elapsed since the mood evaluation data was collected, a particular therapeutic path can be selected out of a set of possible therapeutic paths. By tracking the timing of mood evaluations, the delivery of therapeutic communications can be dynamically tailored to a user's needs.

Aspects and features of the present disclosure include a therapy-providing system capable of providing therapy to a user, such as to monitor, diagnose, and/or treat mental health disorders. While aspects and features of the present disclosure can be used in various environments and for various purposes, the present disclosure can be especially useful when implemented as an artificial intelligence chat-based tool, commonly known as a chatbot. Certain aspects and features of the present disclosure, when implemented with a chat-based tool, provide a quick and easy way for a user to obtain therapy on demand that is dynamically tailored to a user's current and/or past moods, and more specifically to the timing of a user's current and/or past moods.

With traditional, human therapy providers, the provider is able to use their best judgment to gauge whether or not to perform certain therapeutic techniques or check in on the patient's mood. Through an empathic connection with the patient, the provider is able to identify when asking the patient about their mood would be intrusive and unhelpful to the patient's therapy and/or the patient-therapist alliance.

When therapy is attempted in human-computer situations, it can be difficult to build a patient-therapist alliance and maintain the level of trust necessary to achieve therapeutic results. A computer simply cannot connect empathically with a user the same way that a human therapy provider can. However, certain aspects and features of the present disclosure enable a therapy-providing system to mimic certain empathic tendencies by controlling certain therapeutic decisions according to the user's mood (e.g., the type of mood the user is experiencing and the timing of when the user has been experiencing that mood).

The therapy-providing system can obtain mood evaluation data from a user in a provoked (e.g., as a response to a mood evaluation request) or unprovoked (e.g., unsolicited, the user can provide mood evaluation information) fashion. Mood evaluation data can be direct (e.g., “I am happy” equals a “happy” mood) or indirect (e.g., the user's word choice or phrasing is consistent with someone who is happy or with the user's actions when the user has been happy in the past). This mood evaluation data can be timestamped, such that the therapy-providing system can leverage not only the underlying mood evaluation data, but also its timing.

At a later time, immediately after receiving the mood evaluation data or a while after receiving the mood evaluation data, the therapy-providing system can select one out of multiple possible therapeutic paths to take based on the mood evaluation data (e.g., the underlying mood evaluation data and/or its timing). For example, if a user was in a happy mood within the past hour, a first therapeutic path may be taken; if the user was in a sad mood within the past hour, a second therapeutic path may be taken; and if the user's last mood evaluation was over an hour ago, a third therapeutic path may be taken.

A conversation path (which can also be known as a communication path or workflow) can be a set of one or more communications or other engagements between the system and the user. The conversation path can define a set of prompts, statements, or other actions or engagements that can be provided to the user. In some cases, a conversation path can be implemented as a flow chart or a decision tree. A conversation path can have a number of nodes, which can include i) user prompt nodes (e.g., questions or other prompts presented to the user to evoke a response); ii) information nodes (e.g., informational communications provided to the user without expecting a response); iii) input nodes (e.g., unprompted user input); iv) other nodes; or v) any combination of i-iv.

In some cases, a conversation path can be described as having a length defined by a number of nodes associated with the conversation path. The number of nodes can be a set number of nodes in the conversation path, a range of numbers that the conversation path can contain (e.g., if the conversation path includes multiple branches, the minimum of the range may be the shortest path through the conversation path and the largest of the range may be the longest path through the conversation path), or an expected or average number of nodes of the conversation path (e.g., if the conversation path can be between 6-10 nodes, but most users that go through the path do so in 9 nodes, the average number of nodes may be 9). Any suitable technique can be used to obtain an expected or average number of nodes for a conversation path, including using information associated with the user, such as historical data (e.g., from engaging previous conversation paths) and mood related data.

In some cases, different conversation paths can be used to engage the user in different manners, depending on the user's mood. For example, if conversations on a particular topic tend to negatively impact the user's mood when the user is in a first mood state, but positively impact or not impact the user's mood when the user is in a second mood state, the system may select a conversation path that does not include or is not likely to provoke discussion on that topic when the user is in the first mood state, but may select other conversation paths (e.g., conversation paths that include or are likely to provoke discussion on that topic) when the user is in the second mood state. As another example, if it is determined that a user's mood improves more when being asked questions prompting longer responses from the user than when being asked questions prompting short responses from the user, the system may select conversation paths that involve questions prompting longer responses when it is desired to more efficiently improve the user's mood. In yet another example, if it is determined that a user's responses tend to have a first set of traits (e.g., a more complete or detailed response) when the user is in a first mood (e.g., happy and awake) and a second set of traits (e.g., shorter and less engaging responses) when the user is in a second mood (e.g., upset and tired), the system can opt to select conversation paths based on the user's mood with the goal of achieving certain responses from the user. In such an example, if it is desired to obtain a detailed response from the user for a particular question or prompt, the system may avoid a conversation path with that particular question or prompt until it is determined that the user is in the first mood.

In some cases, different conversation paths can be the same other than a manner of engagement. As used herein, a manner of engagement is intended to describe a particular way in which a piece of substantive content can be presented or a substantive goal can be reached. The manner of engagement can be defined by various parameters that do not alter the substantive content or substantive goal. Examples of such parameters include an engagement mode parameter (e.g., defining a mode of engagement, such as via text messages or other short messages, via email, via an app interface or web interface, or the like); a communication style parameter (e.g., defining a style of communication to use, such as full and grammatically correct sentences or shorter statements with abbreviated colloquialisms); a speed parameter (e.g., defining a speed of presenting subsequent communications, such as implementing delays between subsequent communications); a segmentation parameter (e.g., defining an amount of communication segmentation to invoke, if any, such as presenting a full communication when the segmentation parameter is zero or segmenting the full communication into multiple communication segments that are presented sequentially when the segmentation parameter is above zero), and the like.

Thus, in some cases, different conversation paths can be used to relay the same substantive content or achieve the same substantive goal, but in different manners of engagement (e.g., using different modes of communication, different communication styles, different underlying language, and the like). In an example where multiple conversation paths are all associated with a substantive goal of learning about the user's day, when the user is in a first mood, a first conversation path may be used that directly prompts the user “Tell me about your day today,” whereas when the user is in a second mood, a second conversation path may be used that asks the user “What's the most interesting thing you did today?” and when the user is in a third mood, a third conversation path may be used that walks through several prompts in sequence, such as “How was your morning?” “What did you do after lunch today?” and “What are your plans for this evening?” The desired conversation path can be selected to achieve a desired result, such as to make an improvement in the user's mood, to evoke a more thorough or complete response from the user, or to evoke a certain action from the user.

Conversation paths can be used for many systems in which a user is engaged by a chatbot or the like. In a particular subset of user engagement, conversation paths can be used to provide therapy to a user. In such cases, the therapy-providing conversation path can be referred to as a therapeutic path (which can also be known as a therapeutic workflow) can be a set of one or more communications or other engagements between the therapy-providing system and the user. For example, a particular therapeutic path may direct the user through a set of questions to ascertain certain information about the user. In some cases, a therapeutic path can be a therapeutic exercise. A therapeutic exercise can be any set of one or more communications or other engagements between the therapy-providing system and the user that is designed to achieve a certain therapeutic response. Examples of therapeutic exercises or classes of therapeutic exercises include journaling, cognitive restructuring, behavioral activation, sleep optimization, SMART (specific, measurable, achievable, relevant, time-bound) goals support, attention retraining, craving intervention, distraction tool sequencing, mindfulness exercises, distress tolerance, controlled breathing, urge surfing, communication skills training, relationship troubleshooting, grief exercises, loneliness exercises, social skills building, and others.

For illustrative purposes, various examples and description is provided herein with respect to therapeutic paths and therapy-providing systems. However, examples and descriptions specific to therapeutic paths and therapy-providing systems can be altered, as appropriate, for other conversation paths and other systems (e.g., conversation-providing systems). Therefore, reference to therapeutic paths and/or therapy-providing systems as disclosed herein can be replaced, as appropriate and with appropriate adjustment, with other conversation paths and/or conversation-providing systems. For example, while therapeutic paths providing therapeutic exercises are described above, the present disclosure contemplates that other conversation paths can provide other conversation-based exercises, such as such as conversation paths that provide interactive memory game exercises or conversation paths that provide interactive narratives.

In addition to or instead of selecting one of multiple therapeutic paths, the therapy-providing system can make a determination as to whether or not to initiate a subsequent mood evaluation request based on the mood evaluation data (e.g., the underlying mood evaluation data and/or its timing). For example, if a user was in a happy mood within the past hour, the subsequent mood evaluation request may be skipped or otherwise not initiated, but if the user was in a sad mood within the past hour, a subsequent mood evaluation request may be initiated. In another example, if the user had any mood evaluation timestamped within the past hour, the subsequent mood evaluation request may be skipped or otherwise not initiated, but if the user did not have a mood evaluation timestamped within the past hour, the subsequent mood evaluation request may be initiated.

In some cases, determining which therapeutic path to take or whether or not to initiate a mood evaluation request can be based on additional information in addition to mood evaluation data and its timing. Examples of such additional information include determining that a therapy milestone has been achieved (e.g., completion of a therapeutic exercise, starting a therapeutic exercise, or reaching a certain point in a therapeutic path), time of day, preset preferences of the user, and the like.

In some cases, information about the user's mood can be indicative of the likelihood that the user will complete (e.g., fully complete or at least reach a desired milestone of) a given conversation path or therapeutic path. The likelihood that a user would complete a particular path can be represented as a compliance score. Such a compliance score can be calculated based on an algorithm trained using historical compliance data from other users and/or historical compliance data from the given user. Compliance data (e.g., historical compliance data) can include information about the given user (e.g., demographic information), information about a current mood of the user, information about one or more past moods of the user (e.g., the n most recent moods of the user, optionally with timing information), information about the general type or category of path the user is engaging with (e.g., a therapeutic path, a therapeutic path specifically targeted to provide a certain type of therapy, etc.), information about the particular path a user is engaging with, a number of nodes of the path, a number of nodes completed by the user, an indication about whether or not the user completed the path, an indication about whether or not the user fully completed the path, an indication about one or more milestones in the path completed by the user, an indication about which particular node(s) were completed by the user, an indication about which node(s) the user did not complete, an indication about the node during which the user exited or otherwise ceased to complete the path, and/or other data associated with a user engaging in a conversation path.

In some cases, compliance scores can be generated for each of the possible conversation paths available to the user. In some cases, compliance scores can be generated in advance for a range of moods. The compliance scores can be used to select a desired conversation path. For example, out of a grouping of appropriate conversation paths for a certain situation, the conversation path with the highest compliance score may be selected. In another example, if a desired conversation path is to be used, but the compliance score associated with that conversation path is below a threshold value, an alternate conversation path may be selected and/or a separate conversation path can be performed prior to the desired conversation path (e.g., with a goal of improving the user's likelihood that they would complete the desired conversation path).

In some cases, compliance scores can be generated for categories or other groupings of conversation paths (e.g., all conversation paths containing between 5 and 8 nodes; all conversation paths containing a node prompting the user to recount past memories; all conversation paths that are therapeutic paths targeting a particular type of therapy, etc.).

In some cases, information about the user's mood can be used to maximize the likelihood that the user will complete a selected conversation path or therapeutic path. For example, if the user is in an irritated mood, the user may not be interested in answering as many questions or may be unlikely to engage the system for long periods of time, and thus the system may select a path that has fewer nodes (e.g., a shorter path) than would normally be selected if the user were not in the irritated mood. Likewise, if the user is in an especially open, happy, and/or motivated mood, the user may be willing to spend more time engaging the system, and thus the system may select a path that has more nodes (e.g., a longer path) than would normally be selected if the user were not in the open, happy, and/or motivated mood. While the shorter path may be less effective than the longer path (e.g., because it collects less data from the user), it may nevertheless be more effective for a user in an irritated mood to complete the shorter path than to engage and stop a longer path before completing it. In other words, engaging in the longer path but not completing it may be less beneficial (or possibly more detrimental) to the user than engaging in and completing the shorter path.

In some cases, a longer path may include one, some, or all of the nodes of a shorter path plus one or more additional nodes. For example, a therapeutic path may include node(s) asking the user to recount happy experiences in the past day and node(s) asking the user to recount frustrating experiences in the past day. A longer therapeutic path may include these same nodes (e.g., with the same prompts) plus additional node(s), such as node(s) asking if they took any action to reduce their frustration in response to the frustrating experiences. This longer therapeutic path may be more beneficial when the user is in certain moods, since the user may be more likely to complete the longer therapeutic path in those instances.

In some cases, a longer path may include none of the nodes of the alternate shorter path. For example, for the same therapeutic path that includes a handful of nodes asking the user to recount happy experiences and frustrating experiences in the past day, a shorter path may include only a single, different node asking the user to describe any strong emotions they felt in the past day. This shorter path may be more beneficial when the user is in certain moods, since the user may be unlikely to complete the longer therapeutic path (and may become more frustrated) in those instances.

In some cases, user input (e.g., from a response during the conversation path or from a subsequent mood evaluation request) can be used to dynamically update the user's current mood data as the user is engaging in a given path. In some cases, the user's current mood data while the user is engaging in a given path can be used to alter the path. For example, if the user is engaging in a first therapeutic path that normally includes 10 nodes for treating the user, as the user progresses through the therapeutic path the user's responses can be analyzed to identify a shift in mood. This shift in mood can be used to update the user's current mood data. Based on this updated current mood data, the system may alter the therapeutic path such that one or more of those 10 nodes are skipped and/or to add one or more additional nodes. For example, if a user in a frustrated mood is given a shorter therapeutic path due to the higher likelihood that they would complete the therapeutic path, but during the therapeutic path it is determined that the user's mood has sufficiently improved such that they would be likely to complete a longer therapeutic path, the system may alter the therapeutic path to add in additional nodes that would normally have been skipped in the shorter therapeutic path.

Certain aspects and features of the present disclosure relate to a therapy platform that can leverage mood tracking to personalize how therapy is provided to a user. Based on the user's mood and/or the recency of the mood evaluation, the therapy-providing system can alter the tone, content, timing, or other characteristics of how it interacts with the user. As such, the user can be presented with an interactive experience that mimics human empathy, which helps build up the user's trust in the therapy-providing system and helps build up the alliance between the user and the therapy-providing system, which in turn can greatly improve the effects of therapy.

Aspects and features of the present disclosure are associated with the practical application of automatically providing therapy to a user that is dynamically personalized to that user. Certain hardware and software implementations of certain aspects and features of the present disclosure are used to provide particular treatment and/or prophylaxis for mental health disorders. The personalized nature of the provided therapy permits the treatment and/or prophylactic effect to be even stronger than otherwise obtainable in a solely human-to-human interaction.

Aspects and features of the present disclosure provide various improvements to the technological process of human-computer interaction, especially with respect to chatbots, such as therapy chatbots. Examples of such improvements include i) an ability to better track a user's mood and mood timing and their effects on different therapeutic exercises; ii) an ability to dynamically adjust a therapeutic path based on a user's current and/or recent mood(s), which can improve the empathic nature of the chatbot's interactions and strengthen the therapeutic alliance between the human and the computer; iii) an ability to leverage mood-related data to maximize the likelihood that a user will complete a selected conversation path, and iv) an ability to leverage i-iii to provide improved therapy via the chatbot.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

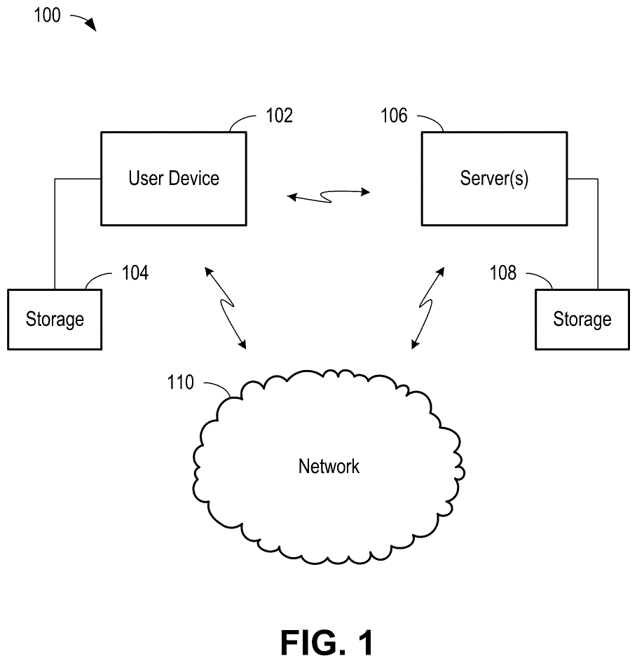

FIG. 1 is a schematic diagram depicting a computing environment 100 according to certain aspects of the present disclosure. The environment 100 can be located in a single physical location or can be distributed about multiple physical locations. Environment 100 can be used to implement a therapy-providing system, such as a chatbot or automated therapy system, such as one being used to receive user input, process the user input to determine a mood evaluation, then select a therapeutic path based on the mood evaluation, such as described in further detail herein. Environment 100 is an example of a suitable environment for implementing the system, although other environments can be used instead.

Environment 100 can include a user device 102 and a server 106, although in some cases one of these devices may not be included. For example, some environments contain only a user device 102. In some cases, multiple user devices 102 can be used. In some cases, other devices can be used. When multiple devices are used, each device can be communicatively coupled together, such as via a direct connection (e.g., a wired connection, such as a universal serial bus (USB) connection, or a wireless connection, such as a Bluetooth connection) or via network 110. Network 110 can be any suitable network, such as a local area network, a wide area network, a cloud, or the Internet. The system can be implemented on a single device (e.g., on user device 102) or can be implemented across multiple devices (e.g., any combination of user device 102 and sever(s) 106).

An individual can interact with the chatbot via the user device 102 and/or another device. Interacting with the chatbot can include i) establishing a chatbot session; ii) receiving prompts or notifications from the chatbot, which can optionally occur outside of an active chatbot session; iii) providing user input, such as free text, constrained text, selections, and the like; iv) receiving therapy, such as in the form of a therapy tool (e.g., therapy exercise) applied by the chatbot as one or more communications (e.g., text entries), including statements or questions; or v) any combination of i-iv.

In some cases, user input can be processed using natural language processing (NLP) techniques to attribute meaning to the provided input. For example, free text user input containing the phrase “This has been a pretty awesome day so far!” may be interpreted by NLP techniques to represent an indication that the user is experiencing a particular mood (e.g., “happiness” or a positive mood), and thus derive certain mood evaluation data.

A user device 102 can act as a primary mode of interaction for one or more individuals to provide user input; receive responses, prompts, and information; and otherwise interact with the system. Examples of user device 102 include any suitable computing device, such as a personal computer, a smartphone, a tablet computer, a smartwatch, a computerized audio recorder, or the like. User device 102 can be operatively coupled to storage 104 to store data associated with applications and processes running on the user device 102. User device 102 can include any combination of input/output (I/O) devices that may be suitable for interacting with the system, such as a keyboard, a mouse, a display, a touchscreen, a microphone, a speaker, an inertial measurement unit (IMU), a haptic feedback device, or other such devices.

One or more servers 106 can be used to enable processes and techniques disclosed herein, such as initiating mood evaluation requests, and receiving user input indicative of mood evaluation data. One or more servers 106 may apply the mood evaluation data for selecting different conversation paths based on the mood evaluation data. One or more of the servers 106 may apply specific applications of the mood evaluation data such as selecting therapeutic paths based on mood evaluation data, generating communications based on the selected therapeutic paths, and transmitting the communications (e.g., for subsequent display on the user device 102). The server(s) 106 can receive user input from user device 102 via network 110, and can provide output (e.g., chatbot output) by transmitting text or other output to the user device 102 via network 110.

Mood evaluation data, mood timestamps, therapy milestone data, therapy milestone timestamps, and other such data can be stored “locally” on a user's user device 102 (e.g., in storage 104) and/or “remotely” on the server(s) 106 (e.g., on storage 108). In some cases, such data can be stored locally, and optionally remotely as well, when it is less than a threshold duration of time old, but can then be stored remotely or only remotely when it is at or greater than the threshold duration of time old. For example, mood evaluation data from the past 1 hour and/or 1 day may be stored locally, whereas mood evaluation data that is older may be stored remotely.

In some cases, various processes and techniques disclosed herein can occur directly on the user device 102, such as selecting a therapeutic path from a set of possible therapeutic paths. In such cases, the user device 102 can provide outputs to the server(s) 106 other than just user input data, such as a selected therapeutic path, which the server(s) 106 may use to generate a communication. However, in many cases, the user device 102 is used to generate a chatbot session with the server(s) 106, transmit user input to the server(s) 106 in response to the user providing the input via an input device (e.g., keyboard or touchscreen), and present (e.g., via an output device like a screen, a speaker, a light, or the like) chatbot outputs (e.g., communications, such as text, images, sounds, or other discernable outputs) in response to receiving communications from the server(s) 106.

In some cases, environment 100 can include additional or fewer components than those depicted.

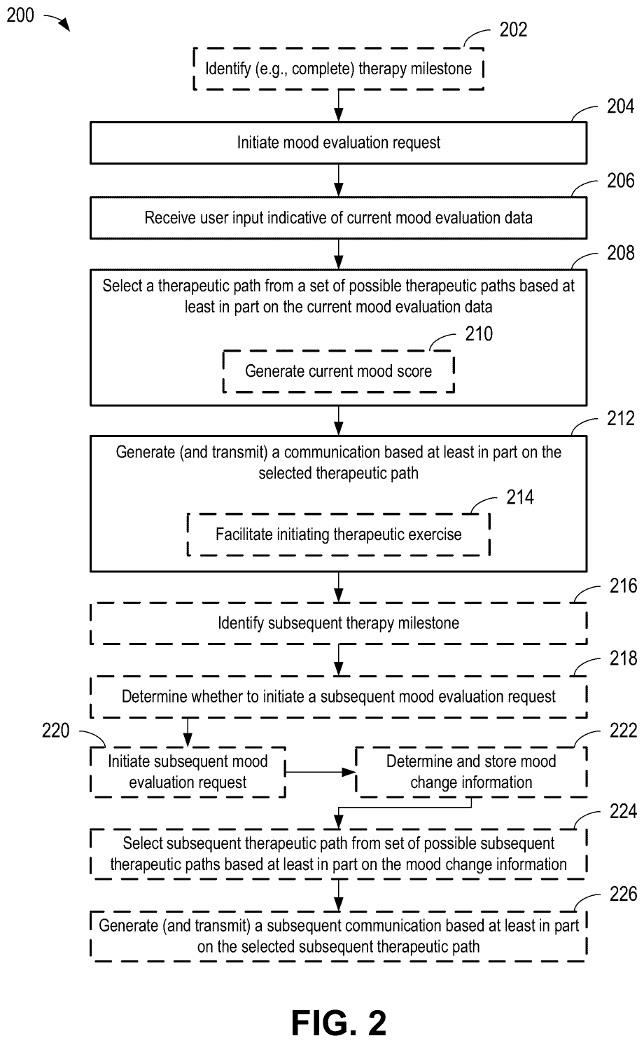

FIG. 2 is a flowchart depicting a process 200 for selecting a therapeutic path, according to certain aspects of the present disclosure. Process 200 can be performed by any suitable computing device or computing devices, such as server(s) 106 of FIG. 1.

At block 204, a mood evaluation request is initiated. Initiating a mood evaluation request can include causing a prompt to be presented to a user that requests information associated with the user's mood. In some cases, the prompt can be a direct question about the user's mood (e.g., “How are you feeling?”). In some cases, the prompt can be an indirect prompt that engages the user in a fashion designed to determine the user's mood (e.g., engaging the user in a communication path and analyzing the user's responses, such as using a machine learning algorithm trained on various users' responses to the same or similar prompts in the communication path). In an indirect example, the user can be asked a series of questions (e.g., “What did you do today?” and “What are you looking forward to tomorrow?” and “What are you thinking about right now?”) and depending on the user's responses, the system can attempt to determine the user's mood. In some cases, initiating a mood evaluation request includes transmitting a communication to a user device (e.g., user device 102 of FIG. 1) that causes the user device to display a question about the user's mood.

In some cases, initiating the mood evaluation request at block 204 can occur i) periodically on a regular basis (e.g., every day at 8 am, every three hours, or the like); ii) automatically in response to a user action (e.g., every time the user opens the app associated with the therapy-providing system, every time the user initiates a chatbot session, or the like); or iii) automatically in response to an identified therapy milestone.

In some cases, at optional block 202, a therapy milestone can be identified, which can cause the mood evaluation request to be initiated at block 204. Identifying a therapy milestone can include identifying that a user has reached a certain place (e.g., a milestone location) in a therapeutic path, such as a start of a therapeutic path (e.g., start of a therapeutic exercise), an end of a therapeutic path (e.g., an end of a therapeutic exercise), a point somewhere between the start and end of a therapeutic path, or an exit point of a therapeutic path (e.g., closing or quitting the therapeutic path before completion). In an example, identifying a therapy milestone can include determining that the user has completed a particular therapeutic exercise or tool.

At block 206, user input is received. The user input can be indicative of current mood evaluation data. The user input can be provided at block 206 in response to the mood evaluation request of block 204. The user input can be a singular response (e.g., a response of “Happy” to a prompt of “How are you feeling?”) or a multi-part response (e.g., a series of responses to a series of questions, such as part of an indirect prompt as disclosed herein).

The mood evaluation data can be a mood, an enumerated mood, a mood score, a mood sign (e.g., a positive mood, a negative mood, or a neutral mood), a mood change (e.g., “Better,” “Worse,” or “About the Same”), or the like. In some cases, the user input can contain the mood evaluation data (e.g., the user input can say “Happy” or can provide a numerical mood score (e.g., 8 out of 10). In some cases, the user input can be analyzed to determine the mood evaluation data (e.g., a response of “Today isn't as bad as yesterday was” can be analyzed, such as using a trained machine learning algorithm, to determine that the user is experiencing a positive mood change).

In some cases, receiving user input at block 206 can include generating a timestamp associated with the user input. The timestamp can be stored in association with the current mood evaluation data. In some cases, the timestamp can be associated with i) the time at which the mood evaluation request was initiated; ii) the time at which the first piece of user input is received; iii) the time at which the last piece of user input in a multi-part response is received; iv) a time denoted by the mood evaluation request (e.g., “How were you feeling this morning” may be associated with a time during that day's morning even if the mood evaluation request and user input were initiated and received in the evening); v) a time denoted by the user input (e.g., “I was feeling much better around noon” may be associated with a time of at or approximately noon, even if the mood evaluation request and user input were initiated and received at a later time); or vi) a time associated with a previously identified therapy milestone, as described with reference to block 202. In some cases, multiple timestamps can be generated and stored in association with the current mood evaluation data.

At block 208, a therapeutic path can be selected from a set of possible therapeutic paths. In some cases, the set of possible therapeutic paths can include at least two therapeutic paths. A therapeutic path can involve engaging the user in a series of communications, which can be designed for a specific therapeutic purpose. In some cases, a given therapeutic paths can include following one or more of the other therapeutic paths, such as after completion of the given therapeutic path. In other words, one possible therapeutic path may involve engaging the user in a first series of communications and a second series of communications, whereas an alternate possible therapeutic path may involve engaging the user in only the second series of communications, effectively skipping the first series of communications.

Selecting the therapeutic path at block 208 can be based at least in part on the current mood evaluation data from block 206. For example, a first therapeutic path may be associated with a first mood and a second therapeutic path may be associated with a second mood, in which case the first therapeutic path is selected if the current mood evaluation data is indicative of the first mood. For example, a first therapeutic path may be associated with a positive mood and a second therapeutic path may be associated with a negative mood. If the current mood evaluation data form block 206 is indicative of a positive mood (e.g., “Happy,” a mood score of 80 out of 100, or simply a positive mood sign), the first therapeutic path can be selected.

In some cases, selecting the therapeutic path at block 208 can optionally include generating a current mood score at block 210. The current mood score at block 210 can be generated based on the current mood evaluation data and optionally one or more previous mood scores. For example, a previous mood score of 70 out of 100 is associated with the user, and the current mood evaluation data is indicative of a “better” mood change, the current mood score may be generated to be some value above 70, such as 80 out of 100. In some cases, generating a current mood score at block 210 can include determining a mood score based on other types of current mood evaluation data and/or the user input from block 206. For example, current mood evaluation data indicative that the user is “happy” can be given a particular mood score, such as a 5 on a scale from −10 to 10. In some cases, generating the current mood score can include applying the user input and/or the current mood evaluation data to a machine learning algorithm trained to output a mood score.

In some cases, selecting the therapeutic path at block 208 can be based at least in part on the timing of mood evaluation data (e.g., based on timestamps associated with the mood evaluation data). For example, a first therapeutic path can be selected if a particular mood evaluation data was experienced within the past hour, whereas a second therapeutic path can be selected if that particular mood evaluation data was experienced outside of the past hour.

In some cases, selecting the therapeutic path at block 208 can be further based on other inputs, such as therapeutic data associated with a therapy milestone (e.g., the therapy milestone of block 202). For example, if the therapy milestone identified at block 202 was completion of a thought-challenging exercise, and the therapeutic data is indicative that the user completed the thought-challenging exercise by identifying and correcting three different thoughts, a particular therapeutic path can be selected. Selecting the therapeutic path can be based on unique combinations of such other inputs and the mood evaluation data (e.g., the underlying mood evaluation data and/or the timing of the mood evaluation data).

In some cases, selecting a therapeutic path at block 208 can include determining one or more compliance scores (e.g., a compliance score for each of the set of possible therapeutic paths) and determining the selected therapeutic path based on the one or more compliance scores. Determining a compliance score can include using the current mood evaluation data (e.g., via a current mood score, although that need not always be the case) to determine a likelihood that the user will complete (e.g., fully complete or at least reach a desired milestone of) a given therapeutic path. In some cases, determining a compliance score can include applying the current mood evaluation data, and optionally other user-related data, to an algorithm trained using historical compliance data of the user and/or historical compliance data of other users.

At block 212, a communication can be generated based at least in part on the selected therapeutic path. Each therapeutic path can include a series of one or more communications (e.g., a communication path) established for a particular therapeutic purpose. For example, a particular therapeutic path may involve the system prompting the user for additional information about why they feel a certain way, in which case one or more communications may be generated, such as “I'm glad you're feeling happy” and “What is making you so happy right now?”

Therapeutic paths can be long (e.g., tens of individual communications or more) or short (e.g., a single communication). A therapeutic path generally involves both sending communications to the user and receiving communications from the user (e.g., user input), although that need not always be the case.

In some cases, generating the communication at block 212 can include optionally facilitating initiating a therapeutic exercise at block 214. Facilitating initiating a therapeutic exercise can include automatically initiating or prompting to initiate (e.g., suggesting to a user) a particular therapeutic exercise. In an example, a particular therapeutic path may involve the system initiating or prompting the user to initiate a particular therapeutic exercise, in which case one or more communications may be generated, such as “I know you are not feeling so great right now. What are you thinking about?” and “Would you like to take a look and see if there are any distortions in your thoughts?” (e.g., to start a therapeutic tool for identifying and correcting distorted thoughts). A therapeutic exercise can be a specific therapeutic path designed to engage the user in a particular exercise designed to achieve a therapeutic result.

In some cases, at optional block 216, a subsequent therapy milestone can be identified. Identifying the subsequent therapy milestone can be similar to identifying the therapy milestone at block 202. Any suitable therapy milestone can be identified, such as starting a therapeutic exercise, ceasing a therapeutic exercise (e.g., completing a therapeutic exercise or exiting a therapeutic exercise before completion), reaching a certain point in a therapeutic path, or otherwise achieving a particular therapy milestone. In some cases, identifying the subsequent therapy milestone at block 216 includes determining that the therapeutic exercise of block 214 has started or ceased.

At optional block 218, in response to identifying the subsequent therapy milestone, a determination can be made as to whether or not to initiate a subsequent mood evaluation request. Determining whether or not to initiate the subsequent mood evaluation request can be based at least in part on the mood evaluation data from block 206. More specifically, the content of the mood evaluation data and/or the timing of the mood evaluation data (e.g., a duration of time between a timestamp associated with the mood evaluation data and the time at which the determination at block 218 is being performed). Timing-based determinations at block 218 can be based on system-set and/or user-set thresholds (e.g., elapsed time must be lower than a threshold value for the subsequent mood evaluation request to be initiated). In some cases, those thresholds can be updated over time based on historical mood evaluation data (e.g., past mood evaluation data extending back by a certain amount of time or number of evaluations).

In an example, a user may complete a particular therapeutic exercise, which can be identified at block 216, causing the determination at block 218 to occur. If the user has not registered any evaluation requests within the past hour, the system can determine that a subsequent mood evaluation request should be performed to obtain another mood evaluation data point. If the user has registered a positive mood evaluation within the past hour, the system can determine that no subsequent mood evaluation request need be performed, since the user is still likely in a positive mood after completing the therapeutic exercise. If the user has registered a negative mood evaluation within the past hour, the system can determine that a subsequent mood evaluation request is to be performed to determine how the user's mood has changed after completing the therapeutic exercise.

In some cases, additional factors can be used to determine whether or not to initiate a subsequent mood evaluation, such as the specific therapy milestone or type of therapy milestone that was identified at block 216, stored preferences associated with the user, and other factors.

If it is determined that a subsequent mood evaluation request is to be performed, the subsequent mood evaluation request can be initiated at block 220. Initiating the subsequent mood evaluation request at block 220 can be the same or similar to initiating the mood evaluation request at block 204.

At optional block 222, mood change information can be determined and stored at block 222. Determining mood change information can include determining subsequent mood evaluation data from user input received in response to the subsequent mood evaluation request, similar to as described with reference to block 206. The subsequent mood evaluation data can be compared to the current mood evaluation data from block 206 to determine how the mood evaluation data has changed over time. For example, current mood evaluation data that includes or is indicative of a mood score of 80 can be compared with subsequent mood evaluation data that includes or is indicative of a mood score of 85 to determine that a positive mood change has occurred.

In some cases, instead of or in addition to determining and storing mood change information at block 222, subsequent mood evaluation data can be determined and stored, such as done with current mood evaluation data at block 206.

At optional block 224, a subsequent therapeutic path can be selected from a set of possible subsequent therapeutic paths based at least in part on the mood change information and/or subsequent mood evaluation data. Selecting a subsequent therapeutic path at block 224 can occur similarly to selecting a therapeutic path at block 208, but using mood change information and/or subsequent mood evaluation data, optionally with current mood evaluation data from block 206.

In response to selecting the subsequent therapeutic path at block 224, one or more subsequent communications can be generated, and optionally transmitted, at block 226, based at least in part on the selected subsequent therapeutic path from block 224. Generating and transmitting a subsequent communication at block 226 can be similar to generating and transmitting a communication at block 212.

Process 200 is depicted with certain blocks in certain orders, although that need not always be the case. In some cases, process 200 can include fewer, additional, or different blocks, and in different orders. For example, in some cases, many additional blocks can occur between blocks 206 and 208. In such an example, a user may provide user input indicative of a current mood evaluation data at 10:00 am, then complete several therapeutic paths and/or therapeutic exercises before reaching block 208 at 10:45 am, wherein the current therapeutic path they are on diverges into multiple possible therapeutic paths from which a single selected therapeutic path will be selected based on the current mood evaluation data from 10:00 am. In another example, process 200 can further include receiving user input associated with a therapeutic path (e.g., user input associated with the user engaging in the therapeutic path, such as responses to prompts that are part of the therapeutic path), updating the current mood evaluation data based on the user input, and altering the therapeutic path (e.g., to remove, skip, redirect, add, or otherwise adjust the nodes that makeup the therapeutic path) based on the updated current mood evaluation data.

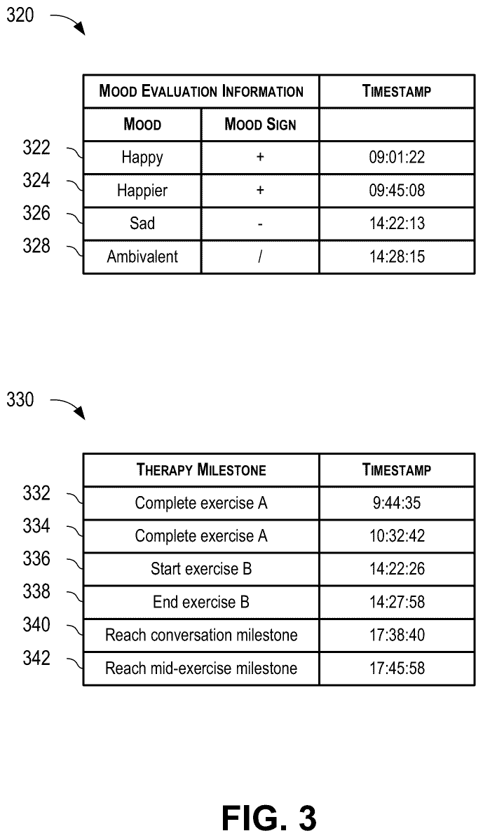

FIG. 3 is a set of tables 320, 330 depicting timestamped mood evaluation information and timestamped therapy milestones, according to certain aspects of the present disclosure. Table 320, 330 can be acquired, such as during execution of process 200 of FIG. 2, and can be stored, such as in storage 104 or storage 108 of FIG. 1. For illustrative purposes, timestamps are shown as hours: minutes: seconds, although actual timestamp data can include fewer or additional information, such as date information and millisecond information.

Table 320 shows timestamped mood evaluation information (e.g., mood evaluation data). Timestamped mood evaluation information can show when a user is experiencing certain moods. Table 320 shows four entries: entry 322 shows that at 09:01:22, the user provided input indicative of a “Happy” mood, which carries a positive mood sign; entry 324 shows that at 09:45:08, the user provided input indicative of a “Happier” mood, which carries a positive mood sign; entry 326 shows that at 14:22:13, the user provided input indicative of a “Sad” mood, which carries a negative mood sign; and entry 328 shows that at 14:28:15, the user provided input indicative of an “Ambivalent” mood, which carries a neutral mood sign.

Table 330 shows timestamped therapy milestone information. Timestamped therapy milestone information can indicate when a certain therapy milestone is reached, such as when a therapeutic exercise has started or ended. Table 330 shows six entries: entry 332 shows that at 9:44:35, the user reached a “Completed exercise A” milestone, indicating the user has completed therapeutic exercise A; entry 334 shows that at 10:32:42, the user reached a “Completed exercise A” milestone, indicating the user has completed another instance of therapeutic exercise A; entry 336 shows that at 14:22:26, the user reached a “Start exercise B” milestone, indicating that the user started therapeutic exercise B at that time; entry 338 shows that at 14:27:58, the user reached a “End exercise B” milestone, indicating that the user ended exercise B (e.g., exited the exercise without completing the exercise); entry 340 shows that at 17:38:40, the user reached a “Reach conversation milestone” milestone, indicating that the user reached a particular point in a conversation path that was important to record in table 330 (e.g., a point in the conversation path that is used to possibly trigger a mood evaluation request); and entry 342 shows that at 17:45:58, the user reached a “Reach mid-exercise milestone” milestone, indicating that the user reached a particular point in a therapeutic exercise that was important to record in table 330 (e.g., a point in the therapeutic exercise that is used to possibly trigger a mood evaluation request).

Tables 320, 330 can show an example user making use of a therapy-providing system as disclosed herein. In this example, the therapy-providing system may have prompted the user during a periodic check-in (e.g., a daily check-in) to provide the mood evaluation at entry 322, at which time the user was happy. A little later in the day, the user may have decided to engage therapeutic exercise A, the completion of which is recorded at entry 332. Upon completing therapeutic exercise A, the therapy-providing system may have prompted the user to provide a mood evaluation, which is recorded at entry 324 (e.g., the user was “Happier” after completing therapeutic exercise A). Later in the day, the user may have engaged therapeutic exercise A again, the completion of which is recorded at entry 334. After completing the therapeutic exercise A again, however, the user may have determined that another mood evaluation request was unnecessary, based at least in part on entry 324 (e.g., because the user recorded a “Happier” mood evaluation within the past hour, and optionally also because the same therapeutic exercise was conducted, the system may determine that another mood evaluation request would be unnecessary and annoying). A bit later, the user may self-record a mood evaluation of “Sad,” which is recorded at entry 326. As a response to that mood evaluation, the therapy-providing system can suggest the user engage therapeutic exercise B, the start of which is recorded at entry 336. The user may not completely finish the exercise, exiting early, which is recorded at entry 338. In response to exiting the therapeutic exercise early, the system can determine that a follow-up mood evaluation request would be beneficial (e.g., because the user had a mood with a negative mood sign within thirty minutes of starting the exercise that was just ended, or because the user had a “Sad” mood within the past hour). The response to this follow-up mood evaluation request is recorded at entry 328. Later that evening, the user may decide to engage the therapy-providing system in a conversation, a milestone of which is recorded at entry 340. The user may then engage the therapy-providing system in a therapeutic exercise, a milestone of which is recorded at entry 342.

Other types of mood evaluation information, therapy milestones, or other mood-related or therapy-related data can be recorded and timestamped. These timestamps can be used to determine the duration of time between any two recorded entries (e.g., the duration of time between entry 234 and 334 is 00:47:34), which can be compared with a threshold (e.g., an upper threshold, a lower threshold, or a range threshold, which can include both an upper threshold and a lower threshold) value when determining subsequent actions to take, such as whether or not to initiate a mood evaluation request or which therapeutic path to select.

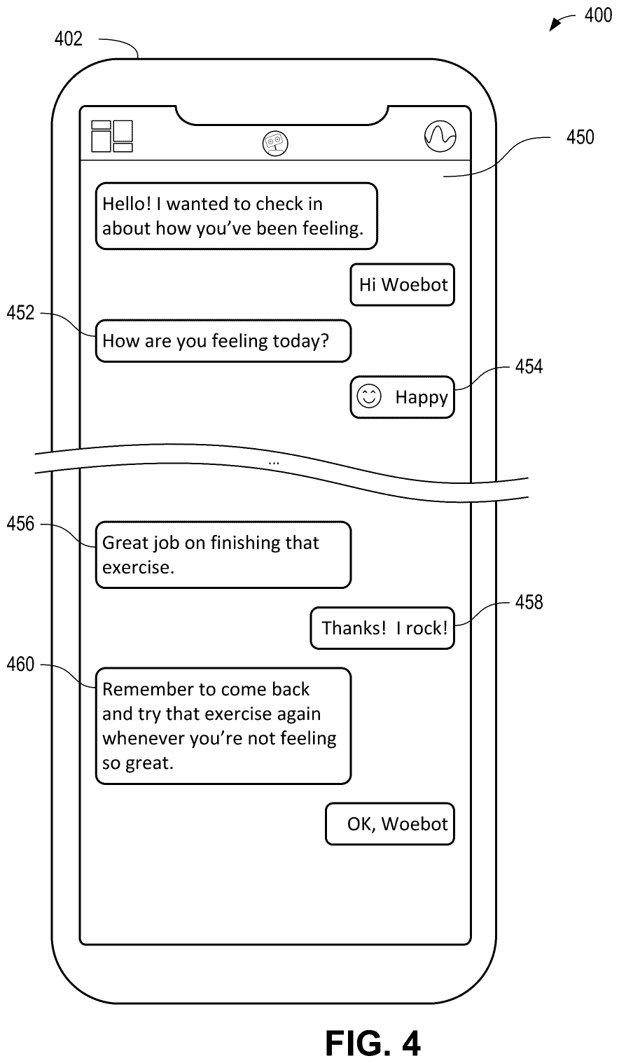

FIG. 4 is a schematic diagram depicting an example conversation path 400 on a graphical user interface 450 for a positive mood evaluation response, according to certain aspects of the present disclosure. The graphical user interface 450 can be presented on a display device of a user device 402 (e.g., a smartphone), such as user device 102 of FIG. 1.

The conversation path 400 can include a mood evaluation request 452 asking the user “How are you feeling today?” The user can provide a mood evaluation response 454, which is shown as a positive mood evaluation response. This mood evaluation response 454 can be stored, along with timestamp information. In some cases, the mood evaluation response 454 can be entered as i) free text; ii) as a selection of one or more responses from a set of constrained responses (e.g., a list of moods from which the user may select one); or iii) any combination of i and ii.

The gap and ellipsis in FIG. 4 are intended to indicate time may pass and additional conversation may occur within the gap. In an example, the user may have engaged a therapeutic tool, the completion of which is acknowledged by the therapy-providing system via communication 456. The user can be presented with a preset response 458 (e.g., a single preset response or a set of possible preset responses from which the user may choose one) that they can use to respond to communication 456. In some cases, the preset response(s) 458 provided for the user to select can be based at least in part on the mood evaluation response 454. For example, because the user's mood evaluation response 454 was a positive one, the preset response 458 can be upbeat and playful.

After the user's preset response 458 is provided, the therapy-providing system can split into multiple possible therapeutic paths. Because the user indicated a positive mood within a certain timeframe via mood evaluation response 454, the therapy-providing system can continue with communication 460. This communication 460 can be selected to not overburden the user with too many and/or unnecessary mood evaluation requests. The communication 460 acts to continue the therapeutic path without asking for another mood evaluation request.

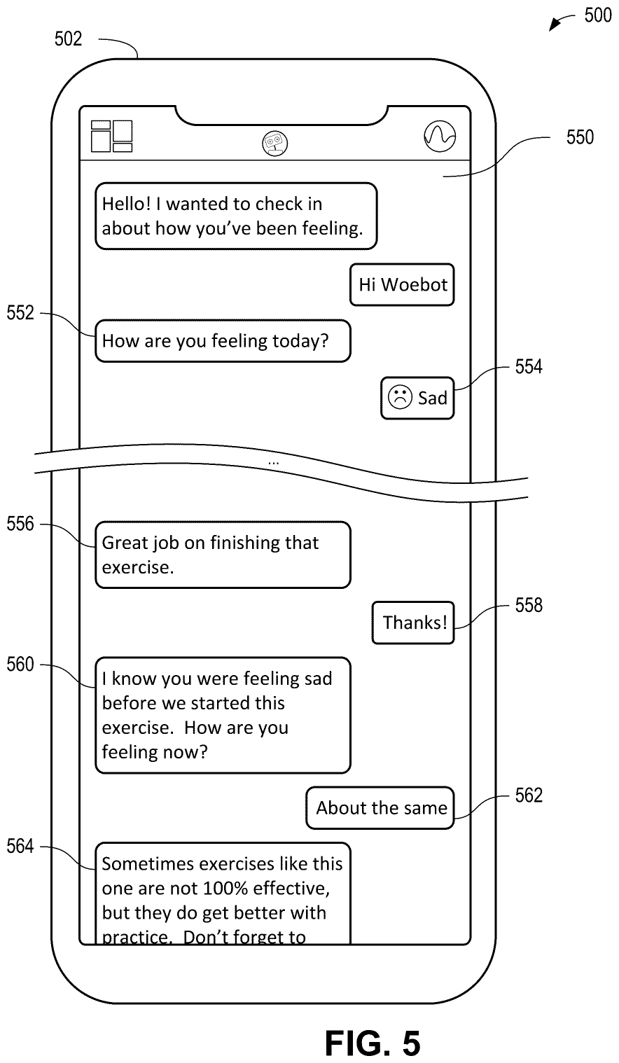

FIG. 5 is a schematic diagram depicting an example conversation path 500 on a graphical user interface 550 for a negative mood evaluation response, according to certain aspects of the present disclosure. The graphical user interface 550 can be presented on a display device of a user device 502 (e.g., a smartphone), such as user device 102 of FIG. 1. Conversation path 500 can be identical to conversation path 400 of FIG. 4, except for a different mood evaluation response 554.

Like in conversation path 400 with respect to FIG. 4, the conversation path 500 can include a mood evaluation request 552 asking the user “How are you feeling today?” The user can provide a mood evaluation response 554, which is shown in FIG. 5 to be a negative mood evaluation response. Similar to FIG. 4, the mood evaluation response 554 can be stored, along with timestamp information.

Similar to FIG. 4, the gap and ellipsis in FIG. 5 indicate passed time and additional conversation, such as the user engaging in a therapeutic tool, the completion of which is acknowledged by the therapy-providing system via communication 556. Similar to FIG. 4, the user can be presented with a preset response 558, although because of the different mood evaluation response 554, the therapy-providing system has generated a different preset response 558. Here, because the user is sad, the preset response 558 is not quite as upbeat and playful as if the user was happy.

After the user's preset response 558 is provided, the therapy-providing system can split into multiple possible therapeutic paths, similar to as described with reference to FIG. 4. Because the user here has indicated a negative mood within a certain timeframe via mood evaluation response 554, the therapy-providing system can continue with a communication 560, which includes a subsequent mood evaluation request. Here, the therapy-providing system has determined that it would be beneficial to obtain another mood evaluation because of the contents and/or timing of the mood evaluation response 554. In response, the user can provide a subsequent mood evaluation response 562. In the example depicted in FIG. 5, the mood changes between the mood evaluation response 554 and the subsequent mood evaluation response 562 is negligible or neutral. Depending on the content of the subsequent mood evaluation response 562 and its timing with respect to the mood evaluation response 554, the therapy-providing system can select one out of a set of possible therapeutic paths. Here, because the mood change is neutral, the therapy-providing system proceeds with communication 564, which is designed to encourage the user to continue engaging in therapeutic exercises even though the user's mood has not improved this time.

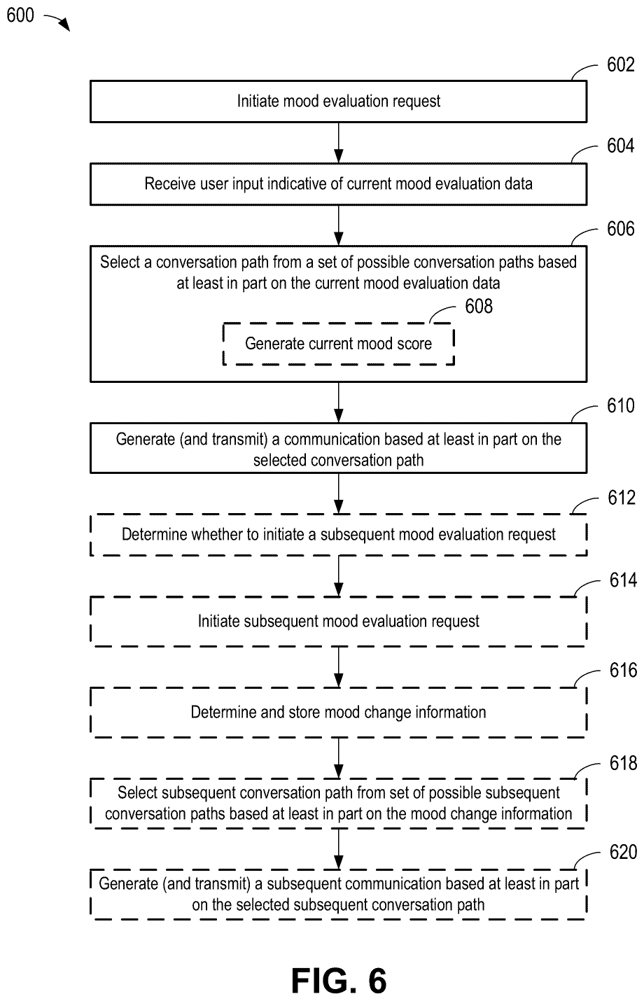

FIG. 6 is a flowchart depicting a process 600 for selecting a conversational path, according to certain aspects of the present disclosure. Process 600 can be performed by any suitable computing device or computing devices, such as server(s) 106 of FIG. 1. In some cases, process 600 can include blocks that are similar to those of process 200 of FIG. 2, but directed to conversation paths that are not necessarily therapeutic paths.

At block 602, a mood evaluation request is initiated. Initiating a mood evaluation request can include causing a prompt to be presented to a user that requests information associated with the user's mood. In some cases, the prompt can be a direct question about the user's mood (e.g., “How are you feeling?”). In some cases, the prompt can be an indirect prompt that engages the user in a fashion designed to determine the user's mood (e.g., engaging the user in a communication path and analyzing the user's responses, such as using a machine learning algorithm trained on various users' responses to the same or similar prompts in the communication path). In an indirect example, the user can be asked a series of questions (e.g., “What did you do today?” and “What are you looking forward to tomorrow?” and “What are you thinking about right now?”) and depending on the user's responses, the system can attempt to determine the user's mood. In some cases, initiating a mood evaluation request includes transmitting a communication to a user device (e.g., user device 102 of FIG. 1) that causes the user device to display a question about the user's mood.

In some cases, initiating the mood evaluation request at block 602 can occur i) periodically on a regular basis (e.g., every day at 8 am, every three hours, or the like); ii) automatically in response to a user action (e.g., every time the user opens the app associated with the therapy-providing system, every time the user initiates a chatbot session, or the like); or iii) automatically in response to an identified therapy milestone.

At block 604, user input is received. The user input can be indicative of current mood evaluation data. The user input can be provided at block 604 in response to the mood evaluation request of block 602. The user input can be a singular response (e.g., a response of “Happy” to a prompt of “How are you feeling?”) or a multi-part response (e.g., a series of responses to a series of questions, such as part of an indirect prompt as disclosed herein).

The mood evaluation data can be a mood, an enumerated mood, a mood score, a mood sign (e.g., a positive mood, a negative mood, or a neutral mood), a mood change (e.g., “Better,” “Worse,” or “About the Same”), or the like. In some cases, the user input can contain the mood evaluation data (e.g., the user input can say “Happy” or can provide a numerical mood score (e.g., 8 out of 10). In some cases, the user input can be analyzed to determine the mood evaluation data (e.g., a response of “Today isn't as bad as yesterday was” can be analyzed, such as using a trained machine learning algorithm, to determine that the user is experiencing a positive mood change).

In some cases, receiving user input at block 604 can include generating a timestamp associated with the user input. The timestamp can be stored in association with the current mood evaluation data. In some cases, the timestamp can be associated with i) the time at which the mood evaluation request was initiated; ii) the time at which the first piece of user input is received; iii) the time at which the last piece of user input in a multi-part response is received; iv) a time denoted by the mood evaluation request (e.g., “How were you feeling this morning” may be associated with a time during that day's morning even if the mood evaluation request and user input were initiated and received in the evening); or v) a time denoted by the user input (e.g., “I was feeling much better around noon” may be associated with a time of at or approximately noon, even if the mood evaluation request and user input were initiated and received at a later time). In some cases, multiple timestamps can be generated and stored in association with the current mood evaluation data.

At block 606, a conversation path can be selected from a set of possible conversation paths. Each conversation path can involve engaging the user in one or more communications, such as a series of communications, which can be designed for a specific purpose. In some cases, a given conversation path can include following one or more of the other conversation paths, such as after completion of the given conversation path. In other words, one possible conversation path may involve engaging the user in a first series of communications and a second series of communications, whereas an alternate possible conversation path may involve engaging the user in only the second series of communications, effectively skipping the first series of communications.

In some cases, selecting a conversation path can include selecting options or parameters for engaging with the user. For example, a first conversation path and a second conversation path may be identical except for one or more altered parameters, such as a topic parameter (e.g., defining one or more topics to use for the communication(s)), a communication style parameter (e.g., defining a style of communication to use, such as full and grammatically correct sentences or shorter statements with abbreviated colloquialisms), an engagement mode parameter (e.g., defining a mode of engagement, such as via text messages or other short messages, via email, via an app interface or web interface, or the like), a speed parameter (e.g., defining a speed of presenting subsequent communications, such as implementing delays between subsequent communications), a segmentation parameter (e.g., defining an amount of communication segmentation to invoke, if any, such as presenting a full communication when the segmentation parameter is zero or segmenting the full communication into multiple communication segments that are presented sequentially when the segmentation parameter is above zero), and the like.

Selecting the conversation path at block 606 can be based at least in part on the current mood evaluation data from block 604. For example, a first conversation path may be associated with a first mood and a second conversation path may be associated with a second mood, in which case the first conversation path is selected if the current mood evaluation data is indicative of the first mood. For example, a first conversation path may be associated with a positive mood and a second conversation path may be associated with a negative mood. If the current mood evaluation data form block 604 is indicative of a positive mood (e.g., “Happy,” a mood score of 80 out of 100, or simply a positive mood sign), the first conversation path can be selected.

In some cases, selecting the conversation path at block 606 can optionally include generating a current mood score at block 608. The current mood score at block 608 can be generated based on the current mood evaluation data and optionally one or more previous mood scores. For example, a previous mood score of 70 out of 100 is associated with the user, and the current mood evaluation data is indicative of a “better” mood change, the current mood score may be generated to be some value above 70, such as 80 out of 100. In some cases, generating a current mood score at block 608 can include determining a mood score based on other types of current mood evaluation data and/or the user input from block 604. For example, current mood evaluation data indicative that the user is “happy” can be given a particular mood score, such as a 5 on a scale from −10 to 10. In some cases, generating the current mood score can include applying the user input and/or the current mood evaluation data to a machine learning algorithm trained to output a mood score.

In some cases, selecting the conversation path at block 606 can be based at least in part on the timing of mood evaluation data (e.g., based on timestamps associated with the mood evaluation data). For example, a first conversation path can be selected if a particular mood evaluation data was experienced within the past hour, whereas a second conversation path can be selected if that particular mood evaluation data was experienced outside of the past hour.

In some cases, selecting a conversation path at block 606 can include determining one or more compliance scores (e.g., a compliance score for each of the set of possible conversation paths) and determining the selected conversation path based on the one or more compliance scores. Determining a compliance score can include using the current mood evaluation data (e.g., via a current mood score, although that need not always be the case) to determine a likelihood that the user will complete (e.g., fully complete or at least reach a desired milestone of) a given conversation path. In some cases, determining a compliance score can include applying the current mood evaluation data, and optionally other user-related data, to an algorithm trained using historical compliance data of the user and/or historical compliance data of other users.

At block 610, a communication can be generated based at least in part on the selected conversation path. Each conversation path can include a series of one or more communications (e.g., a communication path) established for a particular goal. For example, a particular conversation path may involve the system prompting the user for information about their day, in which case one or more communications may be generated, such as “It looks like you're having a good day today” and “What is the most interesting thing that you did today?”

Conversation paths generated at block 610 can be long (e.g., tens of individual communications or more) or short (e.g., a single communication).

The process 600 may include additional evaluations in relation to a selected conversation path to determine whether or not to initiate a subsequent mood evaluation request at optional block 612 (e.g., identifying a potential mood change). For example, information about a user's response to the communication from block 610 can be analyzed to determine whether or not to initiate a subsequent mood evaluation request (e.g., when a potential mood change is identified). Such response information can include information about the content of a user's response, information about a user's response time (e.g., time elasped between transmitting the communication from block 610 and receiving a response from the user), or information about metadata associated with a user's response. In an example, if a user's response contains a word or phrase that is indicative of a possible change in mood, a subsequent mood evaluation request can be initiated. In another example, if a user's response time becomes longer (e.g., longer than a threshold value), it may be determined that the user is likely experiencing a change in mood, in which case a subsequent mood evaluation request can be initiated. In another example, metadata indicating that the user spent a long time drafting a response, pressed the user interface forcefully when typing out the response, or spoke with an angry tone when dictating the response can be used to make a determination that the user is likely experiencing a change in mood, in which case a subsequent mood evaluation request can be initiated.

In some cases, determining whether or not to initiate the subsequent mood evaluation request can be based at least in part on the mood evaluation data from block 604. More specifically, the content of the mood evaluation data and/or the timing of the mood evaluation data (e.g., a duration of time between a timestamp associated with the mood evaluation data and the time at which the determination at block 612 is being performed). Timing-based determinations at block 612 can be based on system-set and/or user-set thresholds (e.g., elapsed time must be lower than a threshold value for the subsequent mood evaluation request to be initiated). In some cases, those thresholds can be updated over time based on historical mood evaluation data (e.g., past mood evaluation data extending back by a certain amount of time or number of evaluations).

If the user has registered a positive mood evaluation within the past hour, the system can determine that no subsequent mood evaluation request need be performed, since the user is still likely in a positive mood after engaging in the selected conversation path. If the user has registered a negative mood evaluation within the past hour, the system can determine that a subsequent mood evaluation request is to be performed to determine how the user's mood has changed after completing part of the conversation path.

If it is determined that a subsequent mood evaluation request is to be performed, the subsequent mood evaluation request can be initiated at block 614. Initiating the subsequent mood evaluation request at block 614 can be the same or similar to initiating the mood evaluation request at block 602.

At optional block 616, mood change information can be determined and stored at block 618. Determining mood change information can include determining subsequent mood evaluation data from user input received in response to the subsequent mood evaluation request, similar to as described with reference to block 602. The subsequent mood evaluation data can be compared to the current mood evaluation data from block 604 to determine how the mood evaluation data has changed over time. For example, current mood evaluation data that includes or is indicative of a mood score of 80 can be compared with subsequent mood evaluation data that includes or is indicative of a mood score of 85 to determine that a positive mood change has occurred.

In some cases, instead of or in addition to determining and storing mood change information at block 616, subsequent mood evaluation data can be determined and stored, such as done with current mood evaluation data at block 604.

At optional block 618, a subsequent conversation path can be selected from a set of possible subsequent conversation paths based at least in part on the mood change information and/or subsequent mood evaluation data. Selecting a subsequent conversation path at block 618 can occur similarly to selecting a conversation path at block 606, but using mood change information and/or subsequent mood evaluation data, optionally with current mood evaluation data from block 604.

In response to selecting the subsequent conversation path at block 618, one or more subsequent communications can be generated, and optionally transmitted, at block 620, based at least in part on the selected subsequent conversation path from block 618. Generating and transmitting a subsequent communication at block 620 can be similar to generating and transmitting a communication at block 610.

Process 600 is depicted with certain blocks in certain orders, although that need not always be the case. In some cases, process 600 can include fewer, additional, or different blocks, and in different orders. For example, in some cases, many additional blocks can occur between blocks 604 and 606. In such an example, a user may provide user input indicative of a current mood evaluation data at 10:00 am, then complete several conversation paths before reaching block 206 at 10:45 am, wherein the current conversation path they are on diverges into multiple possible conversation paths from which a single selected conversation path will be selected based on the current mood evaluation data from 10:00 am. In another example, process 600 can further include receiving user input associated with a conversation path (e.g., user input associated with the user engaging in the conversation path, such as responses to prompts that are part of the conversation path), updating the current mood evaluation data based on the user input, and altering the conversation path (e.g., to remove, skip, redirect, add, or otherwise adjust the nodes that makeup the conversation path) based on the updated current mood evaluation data.

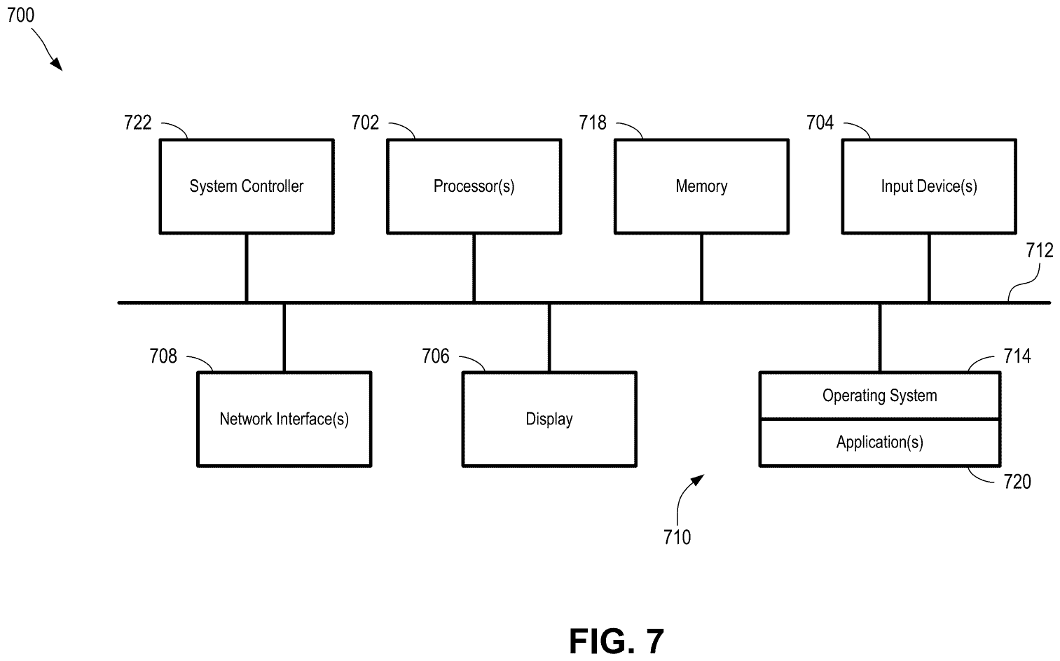

FIG. 7 is a block diagram of an example system architecture 700 for implementing features and processes of the present disclosure, such as those presented with reference to process 200 of FIG. 2 or process 600 of FIG. 6. The architecture 700 can be used to implement a server (e.g., server 106 of FIG. 1), a user device (e.g., user device 102 of FIG. 1), or any other suitable device for performing some or all of the aspects of the present disclosure. The architecture 700 can be implemented on any electronic device that runs software applications derived from compiled instructions, including without limitation personal computers, servers, smart phones, electronic tablets, game consoles, email devices, and the like. In some implementations, the architecture 700 can include one or more processors 702, one or more input devices 704, one or more display devices 706, one or more network interfaces 708, and one or more computer-readable mediums 710. Each of these components can be coupled by bus 712.

Display device 706 can be any known display technology, including but not limited to display devices using Liquid Crystal Display (LCD) or Light Emitting Diode (LED) technology. Processor(s) 702 can use any known processor technology, including but not limited to graphics processors and multi-core processors. Input device 704 can be any known input device technology, including but not limited to a keyboard (including a virtual keyboard), mouse, track ball, and touch-sensitive pad or display. In some cases, audio inputs can be used to provide audio signals, such as audio signals of an individual speaking. Bus 712 can be any known internal or external bus technology, including but not limited to ISA, EISA, PCI, PCI Express, NuBus, USB, Serial ATA or FireWire.

Computer-readable medium 710 can be any medium that participates in providing instructions to processor(s) 702 for execution, including without limitation, non-volatile storage media (e.g., optical disks, magnetic disks, flash drives, etc.) or volatile media (e.g., SDRAM, ROM, etc.). The computer-readable medium (e.g., storage devices, mediums, and memories) can include, for example, a cable or wireless signal containing a bit stream and the like. However, when mentioned, non-transitory computer-readable storage media expressly exclude media such as energy, carrier signals, electromagnetic waves, and signals per se.

Computer-readable medium 710 can include various instructions for implementing operating system 714 and applications 720 such as computer programs. The operating system can be multi-user, multiprocessing, multitasking, multithreading, real-time and the like. The operating system 714 performs basic tasks, including but not limited to: recognizing input from input device 704; sending output to display device 706; keeping track of files and directories on computer-readable medium 710; controlling peripheral devices (e.g., storage drives, interface devices, etc.) which can be controlled directly or through an I/O controller; and managing traffic on bus 712. Computer-readable medium 710 can include various instructions for implementing firmware processes, such as a BIOS. Computer-readable medium 710 can include various instructions for implementing any of the processes described herein, including at least process 200 of FIG. 2 or process 600 of FIG. 6.

Memory 718 can include high-speed random access memory and/or non-volatile memory, such as one or more magnetic disk storage devices, one or more optical storage devices, and/or flash memory (e.g., NAND, NOR). The memory 718 (e.g., computer-readable storage devices, mediums, and memories) can include a cable or wireless signal containing a bit stream and the like. However, when mentioned, non-transitory computer-readable storage media expressly exclude media such as energy, carrier signals, electromagnetic waves, and signals per se. The memory 718 can store an operating system, such as Darwin, RTXC, LINUX, UNIX, OS X, WINDOWS, or an embedded operating system such as VxWorks.

System controller 722 can be a service processor that operates independently of processor 702. In some implementations, system controller 722 can be a baseboard management controller (BMC). For example, a BMC is a specialized service processor that monitors the physical state of a computer, network server, or other hardware device using sensors and communicating with the system administrator through an independent connection. The BMC is configured on the motherboard or main circuit board of the device to be monitored. The sensors of a BMC can measure internal physical variables such as temperature, humidity, power-supply voltage, fan speeds, communications parameters and operating system (OS) functions.

The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language (e.g., Objective-C, Java), including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors or cores, of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computing system that includes a back-end component, such as a data server, or that includes a middleware component, such as an application server or an Internet server, or that includes a front-end component, such as a client computer having a graphical user interface or an Internet browser, or any combination thereof. The components of the system can be connected by any form or medium of digital data communication such as a communication network. Examples of communication networks include, e.g., a LAN, a WAN, and the computers and networks forming the Internet.

The computing system can include clients and servers. A client and server are generally remote from each other and typically interact through a network. The relationship of client and server arises by virtue of computer programs running on the respective computers and having a client-server relationship to each other.

One or more features or steps of the disclosed embodiments can be implemented using an application programming interface (API). An API can define one or more parameters that are passed between a calling application and other software code (e.g., an operating system, library routine, function) that provides a service, that provides data, or that performs an operation or a computation.

The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer will employ to access functions supporting the API.

In some implementations, an API call can report to an application the capabilities of a device running the application, such as input capability, output capability, processing capability, power capability, communications capability, and the like.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art. Numerous changes to the disclosed embodiments can be made in accordance with the disclosure herein, without departing from the spirit or scope of the disclosure. Thus, the breadth and scope of the present disclosure should not be limited by any of the above described embodiments.

Although certain aspects and features of the present disclosure have been illustrated and described with respect to one or more implementations, equivalent alterations and modifications will occur or be known to others skilled in the art upon the reading and understanding of this specification and the annexed drawings. In addition, while a particular feature may have been disclosed with respect to only one of several implementations, such feature may be combined with one or more other features of the other implementations as may be desired and advantageous for any given or particular application.

The terminology used herein is for the purpose of describing particular embodiments only, and is not intended to be limiting of the disclosure. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

One or more elements or aspects or steps, or any portion(s) thereof, from one or more of any of claims 1 to 28 below can be combined with one or more elements or aspects or steps, or any portion(s) thereof, from one or more of any of the other claims 1 to 28 or combinations thereof, to form one or more additional implementations and/or claims of the present disclosure.

As used below, any reference to a series of examples is to be understood as a reference to each of those examples disjunctively (e.g., “Examples 1-4” is to be understood as “Examples 1, 2, 3, or 4”).

Example 1 is a computer-implemented method comprising: initiating a mood evaluation request; receiving user input indicative of current mood evaluation data in response to the mood evaluation request; selecting a conversation path from a set of possible conversation paths based at least in part on the current mood evaluation data, wherein at least a first conversation path of the set of possible conversation paths is selected when the current mood evaluation data is indicative of a first mood and a second conversation path of the set of possible conversation paths is selected when the current mood evaluation data is indicative of a second mood; generating a communication based at least in part on the selected conversation path; and transmitting the communication, wherein the communication, when received, is presented via an output device.

Example 2 is the method of example(s) 1, wherein initiating the mood evaluation request automatically occurs after a period of time following a previous mood evaluation request.

Example 3 is the method of example(s) 1 or claim 2, wherein selecting a conversation path from the set of possible conversation paths based on the current mood evaluation data further includes: generating a current mood score based at least in part on the current mood evaluation data; and selecting the selected conversation path based at least in part on the current mood score.

Example 4 is the method of any one of example(s)s 1 to 3, further comprising: receiving response information associated with a user response to the transmitted communication; determining, based at least in part on the received response information, whether to initiate a subsequent mood evaluation request.

Example 5 is the method of example(s) 4, wherein determining whether to initiate a subsequent mood evaluation request is further based at least in part on the current mood evaluation data.

Example 6 is the method of example(s) 4 or claim 5, wherein determining whether to initiate the subsequent mood evaluation request includes deciding to initiate the subsequent mood evaluation request, the method further comprising: initiating the subsequent mood evaluation request; receiving subsequent user input indicative of subsequent mood evaluation data in response to the subsequent mood evaluation request; determining mood change information using the current mood evaluation data and the subsequent mood evaluation data; storing the mood change information in association with the selected conversation path; selecting a subsequent conversation path from a set of possible subsequent conversation paths based at least in part on the mood change information; generating a subsequent communication based at least in part on the selected subsequent conversation path; and transmitting the subsequent communication, wherein the subsequent communication, when received, is presented via the output device.

Example 7 is the method of any one of example(s)s 4 to 6, further comprising determining an elapsed time since receiving the user input indicative of the current mood evaluation data, wherein determining whether to initiate the subsequent mood evaluation request is further based at least in part on the elasped time.

Example 8 is the method of example(s) 7, wherein determining whether to initiate the subsequent mood evaluation request includes determining to initiate the subsequent mood evaluation request when the current mood evaluation data is indicative of a negative mood and wherein the elapsed time is lower than a threshold elapsed time.

Example 9 is the method of any one of example(s)s 1 to 8, wherein receiving the user input indicative of current mood evaluation data includes storing the current mood evaluation data along with a timestamp of when the user input was received.

Example 10 is the method of any one of example(s)s 1 to 9, further comprising determining an elapsed time since receiving the user input indicative of the current mood evaluation data, wherein selecting the conversation path is further based at least in part on the elapsed time.

Example 11 is the method of any one of example(s)s 1 to 10, further comprising initiating a chatbot session, wherein receiving the user input and transmitting the communication occur via the chatbot session.

Example 12 is the method of any one of example(s)s 1 to 11, wherein the current mood evaluation data is indicative of i) a positive mood, ii) a negative mood, or iii) a neutral mood.

Example 13 is the method of any one of example(s)s 1 to 12, wherein the first conversation path and the second conversation path share common substantive content, and wherein the first conversation path applies a different manner of engagement than the second conversation path.

Example 14 is the method of any one of example(s)s 1 to 13, wherein the first conversation path and the second conversation path share a common substantive goal, and wherein the first conversation path applies a different manner of engagement than the second conversation path.

Example 15 is the method of any one of example(s)s 1 to 14, wherein the selected conversation path is selected to improve a user's mood with respect to the current mood evaluation data.

Example 16 is the method of any one of example(s)s 1 to 15, wherein selecting the conversation path includes not selecting the second conversation path when the current mood evaluation data is not indicative of the second mood.

Example 17 is the method of any one of example(s)s 1 to 16, wherein the first conversation path includes a first number of nodes, wherein the second conversation path includes a second number of nodes, and wherein the first number of nodes is different than the second number of nodes.

Example 18 is the method of example(s) 17, wherein the nodes of the first conversation path include the nodes of the second conversation path and at least one additional node.

Example 19 is the method of example(s) 17 or claim 18, wherein each node of the first number of nodes and the second number of nodes includes a prompt for user input.

Example 20 is the method of any one of example(s)s 1 to 19, wherein selecting the conversation path from the set of possible conversation paths based at least in part on the current mood evaluation data includes: determining, for each conversation path of the set of possible conversation paths, a compliance score based at least in part on the current mood evaluation data, the compliance score being indicative of a likelihood that the user will complete the given conversation path; and determining the selected conversation path based at least in part on the compliance scores.

Example 21 is the method of any one of example(s)s 1 to 20, further comprising: receiving user input associated with the conversation path; updating the current mood evaluation data based at least in part on the user input; and altering the conversation path based at least in part on the current mood evaluation data.

Example 22 is the method of example(s) 21, wherein receiving the user input includes receiving a response to a prompt associated with a node of the conversation path, and wherein altering the conversation path includes, for a given node having a given expected subsequent node, selecting an alternate subsequent node based on the updated current mood evaluation data.

Example 23 is the method of example(s) 21 or claim 22, wherein altering the conversation path includes changing a total number of nodes of the conversation path.

Example 24 is the method of any one of example(s)s 1 to 23, wherein the conversation path includes a decision tree having a plurality of nodes.

Example 25 is a system comprising: one or more processors; and a non-transitory computer-readable storage medium containing instructions which, when executed on the one or more data processors, cause the one or more data processors to perform the method of any one of example(s)s 1 to 24.

Example 26 is a system for dynamically selecting conversation paths, the system configured to implement the method of any one of example(s)s 1 to 24.

Example 27 is a computer program product comprising instructions which, when executed by a computer, cause the computer to carry out the method of any one of example(s)s 1 to 24.

Example 28 is the computer program product of example(s) 27, wherein the computer program product is a non-transitory computer readable medium.

Example 29 is a computer-implemented method comprising: initiating a mood evaluation request; receiving user input indicative of current mood evaluation data in response to the mood evaluation request; selecting a therapeutic path from a set of possible therapeutic paths based at least in part on the current mood evaluation data, wherein at least a first therapeutic path of the set of possible therapeutic paths is selected when the current mood evaluation data is indicative of a first mood and a second therapeutic path of the set of possible therapeutic paths is selected when the current mood evaluation data is indicative of a second mood; generating a communication based at least in part on the selected therapeutic path; and transmitting the communication, wherein the communication, when received, is presented via an output device.

Example 30 is the method of example(s) 29, wherein initiating the mood evaluation request occurs in response to completing a therapy milestone.

Example 31 is the method of example(s) 29, wherein initiating the mood evaluation request automatically occurs after a period of time following a previous mood evaluation request.

Example 32 is the method of any one of example(s)s 29 to 31, wherein selecting a therapeutic path from the set of possible therapeutic paths based on the current mood evaluation data further includes: generating a current mood score based at least in part on the current mood evaluation data; and selecting the selected therapeutic path based at least in part on the current mood score.

Example 33 is the method of any one of example(s)s 29 to 32, further comprising: identifying a subsequent therapy milestone after transmitting the communication; and determining, based at least in part on the current mood evaluation data and in response to identifying the subsequent therapy milestone, whether to initiate a subsequent mood evaluation request.

Example 34 is the method of example(s) 33, wherein identifying the subsequent therapy milestone includes identifying ceasing of a therapeutic exercise.

Example 35 is the method of example(s) 34, wherein transmitting the communication includes initiating the therapeutic exercise or suggesting initiation of the therapeutic exercise.

Example 36 is the method of example(s) 34 or claim 35, wherein determining whether to initiate the subsequent mood evaluation request includes deciding to initiate the subsequent mood evaluation request, the method further comprising: initiating the subsequent mood evaluation request; receiving subsequent user input indicative of subsequent mood evaluation data in response to the subsequent mood evaluation request; determining mood change information using the current mood evaluation data and the subsequent mood evaluation data; storing the mood change information in association with the therapeutic exercise; selecting a subsequent therapeutic path from a set of possible subsequent therapeutic paths based at least in part on the mood change information; generating a subsequent communication based at least in part on the selected subsequent therapeutic path; and transmitting the subsequent communication, wherein the subsequent communication, when received, is presented via the output device.

Example 37 is the method of any one of example(s)s 33 to 36, further comprising determining an elapsed time since receiving the user input indicative of the current mood evaluation data, wherein determining whether to initiate the subsequent mood evaluation request is further based at least in part on the elapsed time.

Example 38 is the method of example(s) 37, wherein determining whether to initiate the subsequent mood evaluation request includes determining to initiate the subsequent mood evaluation request when the current mood evaluation data is indicative of a negative mood and wherein the elapsed time is lower than a threshold elapsed time.

Example 39 is the method of any one of example(s)s 29 to 38, wherein receiving the user input indicative of current mood evaluation data includes storing the current mood evaluation data along with a timestamp of when the user input was received.

Example 40 is the method of any one of example(s)s 29 to 39, further comprising determining an elapsed time since receiving the user input indicative of the current mood evaluation data, wherein selecting the therapeutic path is further based at least in part on the elasped time.

Example 41 is the method of any one of example(s)s 29 to 40, further comprising: identifying a therapy milestone; receiving therapeutic data associated with the therapy milestone; and determining an elapsed time between identifying the therapy milestone and receiving the user input, wherein selecting the therapeutic path is further based at least in part on the therapeutic data and the elapsed time.

Example 42 is the method of example(s) 41, wherein the therapy milestone is completion of a therapeutic exercise and wherein the therapeutic data is based at least in part on a past user input received during the therapeutic exercise.

Example 43 is the method of any one of example(s)s 29 to 42, further comprising initiating a chatbot session, wherein receiving the user input and transmitting the communication occur via the chatbot session.

Example 44 is the method of any one of example(s)s 29 to 43, wherein the current mood evaluation data is indicative of i) a positive mood, ii) a negative mood, or iii) a neutral mood.

Example 45 is the method of any one of example(s)s 29 to 44, wherein the first therapeutic path includes a first number of nodes, wherein the second therapeutic path includes a second number of nodes, and wherein the first number of nodes is different than the second number of nodes.

Example 46 is the method of example(s) 45, wherein the nodes of the first therapeutic path include the nodes of the second therapeutic path and at least one additional node.

Example 47 is the method of example(s) 45 or claim 46, wherein each node of the first number of nodes and the second number of nodes includes a prompt for user input.

Example 48 is the method of any one of example(s)s 29 to 47, wherein selecting the therapeutic path from the set of possible therapeutic paths based at least in part on the current mood evaluation data includes: determining, for each therapeutic path of the set of possible therapeutic paths, a compliance score based at least in part on the current mood evaluation data, the compliance score being indicative of a likelihood that the user will complete the given therapeutic path; and determining the selected therapeutic path based at least in part on the compliance scores.

Example 49 is the method of any one of example(s)s 29 to 48, further comprising: receiving user input associated with the therapeutic path; updating the current mood evaluation data based at least in part on the user input; and altering the therapeutic path based at least in part on the current mood evaluation data.

Example 50 is the method of example(s) 49, wherein receiving the user input includes receiving a response to a prompt associated with a node of the therapeutic path, and wherein altering the therapeutic path includes, for a given node having a given expected subsequent node, selecting an alternate subsequent node based on the updated current mood evaluation data.

Example 51 is the method of example(s) 49 or claim 50, wherein altering the therapeutic path includes changing a total number of nodes of the therapeutic path.

Example 52 is the method of any one of example(s)s 29 to 51, wherein the therapeutic path includes a decision tree having a plurality of nodes.

Example 53 is a system comprising: one or more processors; and a non-transitory computer-readable storage medium containing instructions which, when executed on the one or more data processors, cause the one or more data processors to perform the method of any one of example(s)s 29 to 52.

Example 54 is a system for dynamically selecting therapeutic paths, the system configured to implement the method of any one of example(s)s 29 to 52.

Example 55 is a computer program product comprising instructions which, when executed by a computer, cause the computer to carry out the method of any one of example(s)s 29 to 52.

Example 56 is the computer program product of example(s) 55, wherein the computer program product is a non-transitory computer readable medium.

What is claimed is:

1. A computer-implemented method comprising:
initiating a mood evaluation request;
receiving user input indicative of current mood evaluation data in response to the mood evaluation request;
selecting a therapeutic path from a set of possible therapeutic paths based at least in part on the current mood evaluation data, wherein at least a first therapeutic path of the set of possible therapeutic paths is selected when the current mood evaluation data is indicative of a first mood and a second therapeutic path of the set of possible therapeutic paths is selected when the current mood evaluation data is indicative of a second mood;
generating a communication based at least in part on the selected therapeutic path; and
transmitting the communication, wherein the communication, when received, is presented via an output device.

2. The method of claim 1, wherein initiating the mood evaluation request occurs in response to completing a therapy milestone.

3. The method of claim 1, wherein initiating the mood evaluation request automatically occurs after a period of time following a previous mood evaluation request.

4. The method of claim 1, wherein selecting a therapeutic path from the set of possible therapeutic paths based on the current mood evaluation data further includes:
generating a current mood score based at least in part on the current mood evaluation data; and
selecting the selected therapeutic path based at least in part on the current mood score.

5. The method of claim 1, further comprising:
identifying a subsequent therapy milestone after transmitting the communication; and
determining, based at least in part on the current mood evaluation data and in response to identifying the subsequent therapy milestone, whether to initiate a subsequent mood evaluation request.

6. The method of claim 5, wherein identifying the subsequent therapy milestone includes identifying ceasing of a therapeutic exercise.

7. The method of claim 6, wherein transmitting the communication includes initiating the therapeutic exercise or suggesting initiation of the therapeutic exercise.

8. The method of claim 6, wherein determining whether to initiate the subsequent mood evaluation request includes deciding to initiate the subsequent mood evaluation request, the method further comprising:
initiating the subsequent mood evaluation request;
receiving subsequent user input indicative of subsequent mood evaluation data in response to the subsequent mood evaluation request;
determining mood change information using the current mood evaluation data and the subsequent mood evaluation data;
storing the mood change information in association with the therapeutic exercise;
selecting a subsequent therapeutic path from a set of possible subsequent therapeutic paths based at least in part on the mood change information;
generating a subsequent communication based at least in part on the selected subsequent therapeutic path; and
transmitting the subsequent communication, wherein the subsequent communication, when received, is presented via the output device.

9. The method of claim 5, further comprising determining an elapsed time since receiving the user input indicative of the current mood evaluation data, wherein determining whether to initiate the subsequent mood evaluation request is further based at least in part on the elapsed time.

10. The method of claim 9, wherein determining whether to initiate the subsequent mood evaluation request includes determining to initiate the subsequent mood evaluation request when the current mood evaluation data is indicative of a negative mood and wherein the elapsed time is lower than a threshold elapsed time.

11. The method of claim 1, wherein receiving the user input indicative of current mood evaluation data includes storing the current mood evaluation data along with a timestamp of when the user input was received.

12. The method of claim 1, further comprising determining an elapsed time since receiving the user input indicative of the current mood evaluation data, wherein selecting the therapeutic path is further based at least in part on the elapsed time.

13. The method of claim 1, further comprising:
identifying a therapy milestone;
receiving therapeutic data associated with the therapy milestone; and
determining an elapsed time between identifying the therapy milestone and receiving the user input, wherein selecting the therapeutic path is further based at least in part on the therapeutic data and the elapsed time.

14. The method of claim 13, wherein the therapy milestone is completion of a therapeutic exercise and wherein the therapeutic data is based at least in part on a past user input received during the therapeutic exercise.

15. The method of claim 1, further comprising initiating a chatbot session, wherein receiving the user input and transmitting the communication occur via the chatbot session.

16. The method of claim 1, wherein the current mood evaluation data is indicative of i) a positive mood, ii) a negative mood, or iii) a neutral mood.

17. The method of claim 1, wherein the first therapeutic path includes a first number of nodes, wherein the second therapeutic path includes a second number of nodes, and wherein the first number of nodes is different than the second number of nodes.

18. The method of claim 17, wherein the nodes of the first therapeutic path include the nodes of the second therapeutic path and at least one additional node.

19. The method of claim 17, wherein each node of the first number of nodes and the second number of nodes includes a prompt for user input.

20. The method of claim 1, wherein selecting the therapeutic path from the set of possible therapeutic paths based at least in part on the current mood evaluation data includes:
determining, for each therapeutic path of the set of possible therapeutic paths, a compliance score based at least in part on the current mood evaluation data, the compliance score being indicative of a likelihood that the user will complete the given therapeutic path; and
determining the selected therapeutic path based at least in part on the compliance scores.

21. The method of claim 1, further comprising:
receiving user input associated with the therapeutic path;
updating the current mood evaluation data based at least in part on the user input; and
altering the therapeutic path based at least in part on the current mood evaluation data.

22. The method of claim 21, wherein receiving the user input includes receiving a response to a prompt associated with a node of the therapeutic path, and wherein altering the therapeutic path includes, for a given node having a given expected subsequent node, selecting an alternate subsequent node based on the updated current mood evaluation data.

23. The method of claim 21, wherein altering the therapeutic path includes changing a total number of nodes of the therapeutic path.

24. The method of claim 1, wherein the therapeutic path includes a decision tree having a plurality of nodes.

25. A system comprising:
one or more processors; and
a non-transitory computer-readable storage medium containing instructions which, when executed on the one or more data processors, cause the one or more data processors to perform the method of claim 1.

26. A computer-program product tangibly embodied in a non-transitory machine-readable storage medium, including instructions configured to cause a data processing apparatus to perform the method of claim 1.
